(19) United States
(12) Patent Application Publication
(10) Pub. No.: US 2015/0147305 A1
(43) Pub. Date: May 28, 2015

(54) Asthmatic and Allergenic Nutritional Composition

(71) Applicant: Houn Simon Hsia, Irvine, CA (US)

(72) Inventor: Houn Simon Hsia, Irvine, CA (US)

(21) Appl. No.: 14/088,282

(22) Filed: Nov. 22, 2013

(51) Int. Cl. A61K 36/8998; A61K 36/87; A61K 36/752; A61K 33/04; A61K 31/593; A61K 31/375; A61K 31/355; A61K 31/675; A61K 33/30; A61K 31/202; A61K 31/01; A61K 31/14; A61K 31/122; A61K 31/519; A61K 33/06; A23L 1/29; A61K 36/82

(52) U.S. Cl. CPC A61K 36/8998; A61K 36/82; A61K 36/87; A61K 36/752; A61K 33/04; A61K 31/593; A61K 31/375; A61K 31/355; A61K 31/675; A61K 33/30; A61K 31/202; A61K 31/01; A61K 31/14; A61K 31/122; A61K 31/519; A61K 33/06; A23L 1/296
USPC 424/94.1; 514/690

(57) ABSTRACT

The present invention teaches a combination of specific nutrients suitable for oral consumption by the human body. The compositions of the present invention when orally ingested on a daily basis, work to aid the in slowing or eliminating, or reversing the progression of the respiratory diseases.

It is believed that the key ingredients of the present invention work synergistically together to aid in the slowing, stopping, or reversing the symptoms and metabolic disorder characteristic of the human asthmatic or allergenic condition. The essential components of the present invention are Barley Grass Juice Powder, Green Tea Extract, Grape Seed Extract, Citrus Bioflavonoid, Selenium, Vitamin D3, Vitamin C, Vitamin E, Vitamin B6, Zinc, EPA, DHA, Lycopene, l-Carnitine, Coenzyme Q10, Folic Acid, and Magnesium.

### FIELD OF THE INVENTION

The present invention relates to nutritional compositions useful for treatments to improve the human allergenic and asthmatic conditions.

### BACKGROUND OF THE INVENTION

Asthmatic and Allergenic human conditions are commonplace in today's society. For well over 100 years the industrialization of the world has been on the increase, and since a relationship between industrial air pollution and Asthma and, in general, allergies, has been established, the logical expectation for the future is that the incidence of ailments to the human respiratory system will be on the rise.

Recent studies I have undertaken have shown a relationship between these conditions and changes in relative concentrations of key nutrients known to affect the human immune system. Some of these nutrients are Zinc and Selenium. It has been demonstrated that when trace blood minerals were analyzed in healthy subjects in comparison to asthmatic subjects, stark differences in relative amounts of Zinc to Copper, and Selenium to Copper moieties in the human blood, as well as many antioxidant markers are lowered in the asthmatic subjects in direct comparison to healthy subjects. I have concluded that abnormal distributions of these trace minerals may aggravate oxidative damage that causes inflammation, increased CD4/CD8 lymphocyte ratios, decreased IgE and decreased lung function in asthmatic patients.

It is believed that one of the major causes of the ill-effects of asthma or the allergenic human condition is that it taxes the human immune system, and thus lowers the amounts of key antioxidants as well as reduce the levels of key minerals. A healthy and balanced human immune system is very important to combat the ill-effects of these conditions.

The present invention overcomes many of the shortcomings of treating the asthmatic and allergenic human condition by providing compositions that work together, synergistically, to balance the immune system, and specifically target respiratory ailments including asthmatic conditions.

### SUMMARY OF THE INVENTION

The present invention provides nutritional compositions as powders, lozenges, tablets or liquids, that are employed as oral supplementation to the human diet. The compositions of the present invention provide for supplementation to the diet of the human system, and specifically for those humans predisposed to, or suffering from, the asthmatic, or from the allergenic human condition.

It is the prime objective of the present invention to provide nutritive compositions that provide for an increased activity in combating the human allergenic and asthmatic conditions.

It is another objective of the present invention to provide for compositions to treat the asthmatic or allergenic human condition, and help to reverse the symptoms of the asthmatic or allergenic condition.

### DETAILED DESCRIPTION OF THE INVENTION

The present invention teaches a combination of specific nutrients suitable for oral consumption by the human body. The compositions of the present invention when orally ingested on a daily basis, work to aid the in slowing or eliminating, or reversing the progression of the respiratory diseases.

It is believed that the key ingredients of the present invention work synergistically together to aid in the slowing, stopping, or reversing the symptoms and metabolic disorder characteristic of the human asthmatic or allergenic condition.

A prime essential ingredient included in all the embodiments of the present invention is Green Barley Juice powder. The preferred form of Green Barley Juice powder is derived from organic barley and through the process of lyophilize of the extracted juice of the organic barley. The amount of Green Barley Juice powder 2,500 mg to about 30,000 mg, more preferably from between 5000 mg to about 20000 mg, and most preferably between 10,000 mg to about 18000 mg.

These compositions may also include as preferred essential component the mineral of selenium.

The preferred form of selenium is selenium yeast. The preferred amount of selenium yeast is from about 5 mcg to about 5000 mcg, and more preferred between about 50 mcg to about 1500 mcg, and most preferably from between 200 mcg to about 1000 mcg. The preferred selenium yeast are Saccharomyces cerevisiae, Saccharomyces exiguous, Saccharomyces pastorianus, Saccharomyces boulardii, Saccharomyces bayanus, Saccharomyces eubayanus, Saccharomyces florentinus, Saccharomyces fragilis.

Additionally, Vitamin D3 may be added to the nutritional compositions of the present invention. The preferred form of Vitamin D3 is Cholecalciferol. The preferred amount of Vitamin D3 is from between 50 iu and 5000 iu, and more preferably between from about 200 iu to about 1000 iu, and most preferably from about 150 iu to about 500 iu.

Another nutrient important to the efficacy of the present invention is Vitamin B6. The preferred form of Vitamin B6 is Pyridoxine hydrochloride. The preferred amount is between 0.2 mg to about 100 mg, most preferably between about 0.5 mg to about 50 mg, and most preferably from about 0.75 mg to about 25 mg.

Another nutrient that is important to the present invention is Folic Acid. The preferred amount of Folic Acid is between about 50 mcg to about 5000 mcg, most preferably between about 100 mcg to about 750 mcg, and most preferably between 200 mcg and 400 mcg.

The mineral Zinc is important to include in the present invention. The preferred amount of Zinc is between about 5 mg to about 1500 mg, most preferably between about 5 mg to about 500 mg, and most preferably between 10 mg and 200 mg.

The mineral Magnesium is important to include in the present invention. The preferred form of Magnesium is the oxide. The preferred amount of Magnesium is between about 25 mg to about 2000 mg, most preferably between about 100 mg to about 750 mg, and most preferably between 150 mg and 600 mg.

Additionally, Vitamin E may be added to the nutritional compositions of the present invention. The preferred amount of Vitamin E is from between 15 iu and 1500 iu, and more preferably between from about 30 iu to about 800 iu, and most preferably from about 50 iu to about 400 iu.

Additionally, Vitamin C may be added to the nutritional compositions of the present invention. The preferred amount of Vitamin C is from between 50 mg and 5000 mg, and more preferably between from about 100 mg to about 10000 mg, and most preferably from about 500 mg to about 3000 mg.

Another important nutrient is DHA. The preferred amount of DHA is from about 25 mg to about 15,000 mg, most preferably from between about 50 mg to 2000 mg, and most preferably between about 200 mg to about 1000 mg.

Another important nutrient is EPA. The preferred amount of EPA is from about 25 mg to about 15,000 mg, most preferably from between about 50 mg to 1000 mg, and most preferably between about 300 mg to about 1,500 mg.

Another important nutrient is Lycopene. The preferred amount of Lycopene is from about 0.5 mg to about 30 mg, most preferably from between about 1.5 mg to 20 mg, and most preferably between about 3.0 mg to about 15 mg

Another important nutrient is Coenzyme Q10. The preferred amount of Coenzyme Q10 is from about 10 mg to about 2500 mg, most preferably from between about 25 mg to 12000 mg, and most preferably between about 30 mg to about 300 mg.

Another important nutrient is l-carnitine. The preferred amount of l-carnitine is from about 0.5 mg to about 2000 mg, most preferably from between about 1.0 mg to 200 mg, and most preferably between about 2.0 mg to about 100 mg

Another prime essential ingredient included in all the embodiments of the present invention is Green Tea Extract. The preferred form of Green Tea Extract is a powder that is derived from organic Green Tea. The amount of Green Tea Extract is from about 20 mg to about 500 mg, more preferably from between 50 mg to about 400 mg, and most preferably between 80 mg to about 350 mg.

Another prime essential ingredient included in all the embodiments of the present invention is Grape Seed Extract. The preferred form of Grape Seed Extract is a powder that is derived from organic Grape Seed. The amount of Grape Seed Extract is from about 10 mg to about 200 mg, more preferably from between 30 mg to about 150 mg, and most preferably between 50 mg to about 100 mg.

An additional important nutrient included in the present invention is Citrus Bioflavanoid. The preferred amount of Citrus Bioflavanoid is from about 5 mg to about 200 mg, more preferably from about 10 mg to about 150 mg, and most preferably from about 35 mg to about 100 mg.

The following examples are illustrative only and do not limit the invention in any fashion.

### Example 1

| Ingredient | Amount | |
|---|---|---|
| Barley Grass Juice Powder | 15,000 | mg |
| Inositol | 75 | mg |
| Chromium (from Chromium Yeast) | 150 | mcg |
| Green Tea Extract | 336 | mg |
| Grape Seed Extract | 75 | mg |
| Citrus Bioflavonoid | 54 | mg |
| Selenium (from Selenium yeast) | 300 | mcg |
| Beta Carotene | 21,000 | iu |
| Vitamin A (acetate) | 3000 | iu |
| Vitamin D3 | 50 | iu |
| Vitamin C | 2,031 | mg |
| Vitamin E | 103.1 | iu |
| Vitamin B1 | 4.2 | mg |
| Vitamin B2 | 4.5 | mg |
| Vitamin B3 | 60 | mg |
| Vitamin B6 | 6 | mg |
| Vitamin E (d-tocopherol acetate) | 31.5 | iu |
| Vitamin B12 | 10 | mcg |
| Vitamin E (tocopherol acetate) | 80.6 | iu |
| Folic Acid | 900 | mcg |
| Zinc (gluconate) | 39 | mg |
| Chromium | 150 | mcg |
| Pantothenic acid | 15 | mg |
| EPA | 990 | mg |
| DPA | 660 | mg |
| Biotin | 450 | mcg |
| Calcium (carbonate) | 615 | mg |
| Choline | 45 | mg |
| Magnesium (oxide) | 300 | mg |
| Lycopene | 3 | mg |
| Coenzyme Q10 | 60 | mg |
| l-carnitine | 50 | mg |

The above ingredients were admixed and shaken until homogeneous blend was obtained. The blended composition was poured into 250 mL of water, mixed and then consumed via drinking.

### Example 2

| Ingredient | Amount | |
|---|---|---|
| Barley Grass Juice Powder | 14,500 | mg |
| Inositol | 71 | mg |
| Chromium (from Chromium Yeast) | 140 | mcg |
| Green Tea Extract | 300 | mg |
| Grape Seed Extract | 85 | mg |
| Citrus Bioflavonoid | 54 | mg |
| Selenium (from Selenium yeast) | 300 | mcg |
| Beta Carotene | 21,000 | iu |
| Vitamin A (acetate) | 3000 | iu |
| Vitamin D3 | 300 | iu |
| Vitamin C | 2,031 | mg |
| Vitamin E | 103.1 | iu |
| Vitamin B1 | 4.2 | mg |
| Vitamin B2 | 4.5 | mg |
| Vitamin B3 | 60 | mg |
| Vitamin B6 | 6 | mg |
| Vitamin E (d-tocopherol acetate) | 31.5 | iu |
| Vitamin B12 | 10 | mcg |
| Vitamin E (tocopherol acetate) | 80.6 | iu |
| Folic Acid | 900 | mcg |
| Zinc (gluconate) | 39 | mg |
| Chromium | 150 | mcg |
| Pantothenic acid | 15 | mg |
| EPA | 990 | mg |
| DPA | 660 | mg |
| Biotin | 450 | mcg |
| Calcium (carbonate) | 615 | mg |
| Choline | 45 | mg |
| Magnesium (oxide) | 300 | mg |
| Lycopene | 3 | mg |
| Coenzyme Q10 | 60 | mg |
| l-carnitine | 50 | mg |

The above ingredients were admixed and shaken until homogeneous blend was obtained. The blended composition was poured into 250 mL of water, mixed and then consumed via drinking.

### Example 3

| Ingredient | Amount | |
|---|---|---|
| Barley Grass Juice Powder | 10,000 | mg |
| Inositol | 65 | mg |
| Chromium (from Chromium Yeast) | 130 | mcg |
| Green Tea Extract | 336 | mg |
| Grape Seed Extract | 75 | mg |
| Citrus Bioflavonoid | 54 | mg |
| Selenium (from Selenium yeast) | 350 | mcg |
| Beta Carotene | 20,000 | iu |
| Vitamin A (acetate) | 2900 | iu |
| Vitamin D3 | 400 | iu |
| Vitamin C | 2,000 | mg |
| Vitamin E | 100 | iu |
| Vitamin B1 | 3.2 | mg |
| Vitamin B2 | 4.5 | mg |
| Vitamin B3 | 50 | mg |
| Vitamin B6 | 5 | mg |
| Vitamin E (d-tocopherol acetate) | 30.5 | iu |
| Vitamin B12 | 10 | mcg |
| Vitamin E (tocopherol acetate) | 80.6 | iu |
| Folic Acid | 900 | mcg |
| Zinc (gluconate) | 39 | mg |
| Chromium | 150 | mcg |
| Pantothenic acid | 15 | mg |
| EPA | 900 | mg |
| DPA | 600 | mg |
| Biotin | 431 | mcg |
| Calcium (carbonate) | 605 | mg |
| Choline | 37 | mg |
| Magnesium (oxide) | 320 | mg |
| Lycopene | 3.5 | mg |
| Coenzyme Q10 | 60 | mg |
| l-carnitine | 50 | mg |

The above ingredients were admixed and shaken until homogeneous blend was obtained. The blended composition was poured into 250 mL of water, mixed and then consumed via drinking.

What is claimed is:

1. A nutritional composition comprising Barley Grass Juice Powder, Green Tea Extract, Grape Seed Extract, Citrus Bioflavonoid, Selenium, Vitamin D3, Vitamin C, Vitamin E, Vitamin B6, Zinc, EPA, DHA, Lycopene, l-Carnitine, Coenzyme Q10, Folic Acid, and Magnesium.

2. The composition according to claim 1 where the amount of Barley Grass Juice Powder is from about 2,500 mg to about 30,000 mg.

3. The composition according to claim 1 where the amount of Green Tea Extract is from about 20 mg to about 500 mg.

4. The composition according to claim 1 where the amount of Grape Seed Extract is from about 10 mg to about 200 mg.

5. The composition according to claim 1 where the amount of Citrus Bioflavonoid is from about 5 mg to about 200 mg.

6. The composition according to claim 1 where the amount of Selenium is from about 30 mcg to about 3000 mcg.

7. The composition according to claim 1 where the amount of Vitamin D3 is from about 50 iu to about 2000 iu.

8. The composition according to claim 1 where the amount of Vitamin C is from about 50 mg to about 15000 mg.

9. The composition according to claim 1 where the amount of Vitamin E is from about 15 iu to about 1500 iu.

10. The composition according to claim 1 where the amount of Vitamin B6 is from about 0.5 mg to about 50 mg.

11. The composition according to claim 1 where the amount of Zinc is from about 5 mg to about 150 mg.

12. The composition according to claim 1 where the amount of DHA is from about 25 mg to about 15000 mg.

13. The composition according to claim 1 where the amount of EPA is from about 25 mg to about 15000 mg.

14. The composition according to claim 1 where the amount of Lycopene is from about 0.5 mg to about 30 mg.

15. The composition according to claim 1 where the amount of l-Carnitine is from about 0.5 mg to about 2000 mg.

16. The composition according to claim 1 where the amount of Coenzyme Q10 is from about 10 mg to about 2500 mg.

17. The composition according to claim 1 where the amount of Folic Acid is from about 50 mcg to about 5000 mcg.

18. The composition according to claim 1 where the amount of Magnesium is from about 25 mg to about 2000 mg.
